(19) United States
(12) Patent Application Publication
(10) Pub. No.: US 2014/0350517 A1
(43) Pub. Date: Nov. 27, 2014

(54) INJECTION DEVICE WITH STRESS PROTECTION

(71) Applicant: Allergan, Inc., Irvine, CA (US)

(72) Inventors: Zachary P. Dominguez, Santa Barbara, CA (US); Tiago Bertolote, Geneve (CH); Ethan Franklin, Goleta, CA (US); Justin J. Schwab, Santa Barbara, CA (US); Mike Augarten, Goleta, CA (US); Jason Metzner, Covington, WA (US)

(73) Assignee: Allergan, Inc., Irvine, CA (US)

(21) Appl. No.: 14/284,268

(22) Filed: May 21, 2014

Related U.S. Application Data
(60) Provisional application No. 61/826,791, filed on May 23, 2013.

(51) Int. Cl. A61M 5/315; A61M 5/50

(52) U.S. Cl. CPC A61M 5/31566; A61M 5/31501; A61M 5/5086; A61M 2005/3128
USPC 604/506; 604/218; 604/189; 604/220; 604/111

(57) ABSTRACT

Described are devices and systems for protection of injectable substances such as adipose cells from excessive stress or pressure during injection procedures. Further described are stress control mechanisms for detecting and/or controlling stress or pressure within injection devices.

### CROSS REFERENCE TO RELATED APPLICATIONS

This application claims the benefit of U.S. provisional patent application No. 61/826,791, filed May 23, 2013, the entire disclosure of which is incorporated herein by reference.

### FIELD

The present description generally relates to medical injection devices and more specifically relates to a medical syringe designed for stress protection of injectable substances.

### BACKGROUND

Injection of liquids, gels, and gases through a syringe is common practice in many medical applications. In certain circumstances, the injectable substance is susceptible to damage from the injection process itself, for example, as a result of excessive pressure in the syringe or shear forces during forced extrusion of the substance through the cannula.

Such injectables subject to possible damage include those containing living tissue, for example, living cells, for example, adipose tissue cells being transplanted from one area of the body to another. Such cells must remain healthy and viable upon transplant in order for the graft to be successful.

Injection of living tissue, as with autologous fat grafting procedures, requires particular care in protecting cells from damage. In the past, much of the protection of these substances has been focused on preserving viability of the cells in the early stages of the transplant procedure, for example, before the cells even enter a syringe for injection into a host site. For example, numerous procedures have been proposed and implemented in the industry which are primarily aimed at processing newly explanted tissue to separate cells from other components of the tissue, by centrifuging the tissue, washing the separated cells and storing the processed cells to maintain the cells in an environment conducive to preserving viability.

There is a need for better devices, mechanisms, systems and methods for injecting these fragile substances into the body to improve the outcome of the procedure.

### SUMMARY

The present disclosure is directed, at least in part, toward devices and systems for maintaining the integrity of injectable substances during an injection procedure. For example, the systems may be directed toward maintaining cell viability during a transplant procedure, for example, by protecting the tissue from excessive stresses that may be caused by injection of the cells through a syringe and cannula and into the body.

In one embodiment, an injection device comprises a syringe having a distal end connectable to a cannula, the syringe comprising a barrel suitable to contain an injectable substance, and a plunger, movable within the barrel for forcing the injectable substance through the syringe distal end; a cannula disposed in the distal end of the syringe; and a stress control mechanism configured to detect, control, or detect and control a pressure within the barrel to reduce potential for damaging the injectable substance.

In another embodiment, the stress control mechanism is a visual feedback indicator configured to detect a pressure within the barrel and display visual feedback to the user. In some embodiments, the visual feedback indicator is a continuous visual feedback indicator or a discrete visual feedback indicator.

In some embodiments, the visual feedback indicator comprises: a cylinder having a distal end in communication with fluid pressure in the barrel; a spring-loaded plunger movable within the cylinder; and an indicator configured to display a pressure reading based on the position of the plunger. In another embodiment, the visual feedback indicator comprises photoelastic material disposed between two polarizers; or a binary snap indicator configured to maintain one configuration when the pressure is below a predetermined level and to change to a second configuration when the pressure reaches or exceeds the predetermined level.

In some embodiments, the visual feedback indicator comprises a plunger assembly comprising: a plunger rod comprising a distal end and a proximal end; a plunger tip extending through the plunger rod, wherein the plunger tip comprises a distal end comprising a head and a proximal end comprising an indicator configured to display a pressure reading based on the position of the plunger tip; a spring disposed between the plunger tip head and the distal end of the plunger rod, wherein the plunger tip is movable within the plunger rod.

In another embodiment, the stress control mechanism is a tactile feedback indicator configured to detect a pressure within the barrel and provide tactile feedback to the user.

In one embodiment, the tactile feedback indicator comprises a plunger assembly comprising: a plunger rod comprising a distal end and a proximal end; a plunger tip extending through the plunger rod, wherein the plunger tip comprises a distal end comprising a head and a proximal end comprising a tactile indicating element; a spring disposed between the plunger tip head and the distal end of the plunger rod; wherein the plunger tip is movable within the plunger rod.

In another embodiment, the stress control mechanism comprises a hard stop mechanism configured to prevent the pressure within the barrel from exceeding a predetermined level. For example, in one embodiment, the plunger comprises a distal end and a proximal end, wherein the hard stop mechanism comprises a deformable bumper disposed between the proximal end of the plunger and the distal end of the plunger, wherein the deformable bumper is configured to exert a frictional force against the barrel when the pressure within the barrel exceeds a predetermined level, and wherein the frictional force prevents the plunger from moving within the barrel.

In another embodiment, the hard stop mechanism comprises a plunger assembly. The plunger assembly can include: a plunger rod comprising a distal end and a proximal end; a plunger tip disposed within the barrel; and a series of mechanical linkages connected by one or more springs disposed between the distal end of the plunger rod and the plunger tip. In some embodiments, the plunger assembly can be movable within the barrel when the pressure within the barrel is below a predetermined level. In other embodiments, the mechanical linkages may prevent movement of the plunger assembly by contacting the barrel when the pressure within the barrel reaches or exceeds the predetermined level.

In another embodiment, the stress protection mechanism comprises an electromechanical sensor and indicator, which may be configured to produce a visual, aural, tactile, or vibrational indication when the pressure within the barrel reaches or exceeds a predetermined level. In a further embodiment, the electromechanical sensor and indicator comprises at least one light emitting diode (LED). In a further embodiment, the electromechanical sensor and indicator comprises: electronic components, a printed circuit board, a compliant interface, a sensor, an indicator, and a battery or piezoelectric element.

In another embodiment, the hard stop mechanism comprises a plunger assembly comprising: a plunger rod comprising a distal end and a proximal end; a plunger tip disposed within the barrel; a compartment disposed between the distal end of the plunger rod and the plunger tip, wherein the compartment comprises magnetorheological fluid; and a force sensor and an electronic circuit disposed between the distal end of the plunger rod and the compartment; wherein the electronic circuit produces a magnetic field when the force sensor detects a pressure within the barrel that reaches or exceeds a predetermined level.

In another embodiment, the stress control mechanism comprises a pressure release valve configured to open and release pressure when the pressure within the barrel reaches or exceeds a certain level, or a reverse pressure release valve configured to close when the pressure within the barrel reaches or exceeds a certain level.

In a further embodiment, an injection device may comprise a second stress protection mechanism configured to detect, control, or detect and control a pressure within the barrel to reduce potential for damaging the injectable substance. In another embodiment, the first and second stress protection mechanisms are different; for example, in one embodiment, the first stress protection mechanism is configured to detect a pressure within the barrel, and the second stress protection mechanism is configured to control a pressure within the barrel.

In a further embodiment, an injection device may comprise an injectable substance within the barrel. In some embodiments, the injectable substance can comprise cells such as adipose cells.

In still another embodiment, provided is a method for administering an injectable substance to a subject. The method can include the steps of: pushing the plunger rod within the barrel of an injection device as described herein to inject the injectable substance into a target site in the subject; and stopping the pushing when the stress control mechanism indicates that a predetermined level of pressure in the barrel is reached, or when the stress control mechanism prevents further movement of the plunger rod within the barrel. In another embodiment, the method may further include the step of loading the injectable substance into an injection device as described herein. In other embodiments, the injection device may be provided pre-loaded with the injectable substance. In still further embodiments, the method may include the steps of resuming pushing the plunger rod within the barrel of the injection device after the stress control mechanism indicates that the stress or pressure within the barrel is below the predetermined level, or after the stress or pressure is released and the stress control mechanism allows further movement of the plunger rod within the barrel.

### BRIEF DESCRIPTION OF THE DRAWINGS

The present description may be more clearly understood and the advantages thereof better appreciated by considering the below Detailed Description and accompanying Drawings of which:

FIGS. 1A-1B illustrate an injection device including an exemplary stress protection mechanism comprising a visual feedback indicator device as part of the device (FIG. 1A) or as part of an accessory (FIG. 1B).

FIG. 2 illustrates an exemplary visual feedback indicator device.

FIGS. 3A-3C illustrate exemplary changes in the visual feedback indicator device as pressure is generated in the system.

FIGS. 4A-4C illustrate an injection device including an exemplary stress protection mechanism comprising photoelastic material.

FIGS. 5A-5B illustrate an exemplary stress protection mechanism comprising a snap visual indicator.

FIGS. 6A-6C illustrate changes in an exemplary stress protection mechanism comprising a snap visual indicator as pressure is applied to the injection system.

FIG. 7 illustrates an injection device including an exemplary stress protection mechanism comprising a tactile feedback mechanism.

FIG. 8 illustrates exemplary use of an injection device including a stress protection mechanism comprising a tactile feedback mechanism.

FIGS. 9A-9C illustrate changes in an exemplary stress protection mechanism comprising a tactile feedback mechanism as pressure is applied to the injection system.

FIG. 10 illustrates an injection device including an exemplary stress protection mechanism comprising a tactile feedback mechanism.

FIG. 11 illustrates the separate construction of the plunger rod and plunger tip in an injection device including an exemplary stress protection mechanism comprising a tactile feedback mechanism.

FIG. 12 illustrates an injection device including an exemplary stress protection mechanism comprising a continuous visual feedback indicator.

FIGS. 13A-13C illustrate changes in an exemplary stress protection mechanism comprising a continuous visual feedback indicator as pressure is applied to the injection system.

FIGS. 14A-14C illustrate changes in an exemplary stress protection mechanism comprising a bumper as pressure is applied to the injection system.

FIGS. 15A-15B illustrate changes in an exemplary stress protection mechanism comprising a bumper as pressure is applied to the injection system.

FIGS. 16A-16C illustrate changes in an exemplary stress protection mechanism comprising a mechanical hard stop as pressure is applied to the injection system.

FIG. 17 illustrates an injection device including an exemplary stress protection mechanism comprising an electromechanical sensor.

FIG. 18 illustrates an exemplary stress protection mechanism comprising an electromechanical sensor.

FIG. 19 illustrates an exemplary stress protection mechanism comprising an electromechanical sensor which includes a piezoelectric element.

FIG. 20 illustrates an injection device including an exemplary stress protection mechanism comprising a pressure release valve.

FIG. 21 illustrates an injection device including an exemplary stress protection mechanism comprising a reverse pressure release valve.

FIG. 22 illustrates an injection device including an exemplary stress protection mechanism comprising a reverse pressure relieve valve.

FIG. 23 illustrates an injection device including an exemplary stress protection mechanism comprising pressure film and a plunger rod configured to include a structural feature that translates injection force to side load against the wall of the device.

FIG. 24 illustrates an exemplary injection profile in a pressure film indicator after an injection procedure using the device illustrated in FIG. 23.

FIG. 25 illustrates an injection device including an exemplary stress protection mechanism comprising magnetorheological fluid.

### DETAILED DESCRIPTION

The systems described herein are generally configured to inject substances, particularly fragile substances, into the body of a subject while minimizing the amount of stress or pressure the substances are subjected to during injection, in order to prevent damage to the substances.

The present disclosure provides systems, devices, and mechanisms which addresses these and other issues related to injection of such substances, for example, injectable products which include living cells. While not being limited by mechanism of action, it is believed that damage may occur to such fragile injectables as a result of stresses (both normal and shear) exerted on the injectable that exceed certain threshold, or maximum allowable, stresses. In the scope of this disclosure, the terms “product”, “injectable”, “substance”, “composition”, and “material”, as well as combinations of these terms, are sometimes used interchangeably, and are generally used to identify fragile injectable substances that could become damaged during an injection procedure as a result of stresses in a syringe or other injector device.

For example, in some embodiments, adipose tissue for use in cosmetic fat grafting procedures is harvested or removed from the body, for example, by a suction device, from one area of the patient's body. In some embodiments, the fat may be harvested from a location where excess fat is located, such as the abdomen or thighs of the patient. The harvested material, sometimes referred to as “lipoaspirate”, contains adipose cells (adipocytes), other cells, oils from damaged cells, intracellular materials and tumescent fluid that was used to infuse the donor area to facilitate the harvesting procedure. In some embodiments, the lipoaspirate may then be processed to separate the living cells from these other components. In further embodiments, the cell-containing material may then be treated in some manner, and/or may be mixed with other materials, for example, to enhance cell viability. During the fat grafting steps of the procedure, the processed or unprocessed adipose tissue, or the separated cellular component thereof, is then reintroduced into one or more different areas of the same patient, for example, into the breasts. This is typically performed by injection of the material in a suitable region of the breast, for example, with the goal of creating more volume in the breast, or modifying the shape or firmness of the breast. In other embodiments, the grafting procedure may be performed in any other areas of the body where additional volume or shaping is desired, for example, the face, neck, hands, or buttocks, as well as the skin, for example, to reduce depressions, divots, or wrinkles in the skin.

It can be appreciated that, like all living cells, adipose cells are fragile and can easily become damaged by mechanical stresses, particularly when the cells have been manipulated and removed from their natural location in the body. Adipocytes are particularly susceptible to damage from exposure to both normal and shear forces. Excessive damage to the transplanted adipose cells may reduce the chance of success of the fat grafting procedure.

The present disclosure provides systems and devices for reducing the potential for damage to injectable substances, including cells such as adipocytes, by reducing exposure of the cells to mechanical stresses in an injector syringe.

In a broad aspect, an injection system or device is provided, generally comprising a syringe having a distal end connectable to a needle or cannula, and further comprising at least one mechanism for controlling stressors in the syringe and/or cannula. The syringe may comprise a barrel for containing an injectable substance, and a hand actuatable plunger, movable in the barrel, for forcing the injectable substance through the barrel and out through an outlet in a distal tip of the cannula.

As used herein, the terms “mechanism for controlling stressors”, “stress control mechanism”, and the like refer to a mechanism or device coupled to or associated with an injection system or device as described herein. A stress control mechanism as described herein may be active or passive, or it may have both active and passive properties. A passive stress control mechanism can generally provide information about the system but does not modify the system. Thus, a passive stress control mechanism, may, for example, detect stress or pressure in the system and convey the level of stress or pressure in the system to the user (e.g., by visual, aural, tactile, or vibrational means), so that the user can respond by modifying use of the system in order to prevent damage to the injectable substance. For example, if a stress control mechanism indicates a certain level of pressure in the system, the user can slow or stop the injection process until the pressure is decreased (e.g., if the system becomes clogged, the user could stop the injection process, remove the clogged material, and thereafter resume the injection process). An active stress control mechanism detects stress or pressure in the system and responds, when the stress or pressure reaches a certain level, by modifying the system itself, without input from the user. Thus, active stress control mechanisms as described herein may, for example, detect stress or pressure in the system and modify the system to prevent the stress or pressure from increasing. In some embodiments, if the stress control mechanism detects a certain level of pressure in the system, the stress control mechanism can act to prevent further input of pressure into the system by the user. For example, if a stress control mechanism detects a certain level of pressure in the system, the stress control mechanism may act as a brake to stop the plunger from any further movement, even if the user continues to push on the plunger. In other embodiments, if an active stress control mechanism detects a certain level of pressure or stress in the system, the stress control mechanism can act to release pressure (e.g., through a valve) so that the pressure in the system cannot rise above a certain level.

In some embodiments, a stress control mechanism as used herein may have both active and passive properties. For example, a single stress control mechanism may have active properties as described above, and may also provide information about the level of stress or pressure to the user (e.g., by visual, aural, tactile, or vibrational means). In other embodiments, an injection system may comprise more than one stress control mechanism. For example, an injection system may comprise separate passive and active stress control mechanisms. In other embodiments, an injection system as described herein may comprise multiple active stress control mechanisms or multiple passive stress control mechanisms. In still other embodiments, an injection system may have three or more total stress control mechanisms.

In some embodiments, a stress control mechanism may be provided as part of the injection system (e.g., physically integrated into the injection system or irreversibly attached) or may be provided as a removable or detachable accessory.

Accordingly, in one embodiment, the injection system includes a mechanism which is generally configured to monitor or control pressure in the injection system, thereby reducing normal stresses on a product being moved through the injection system, for example during injection of the product into a target region of a subject.

In some embodiments, the subject is an animal. In one embodiment, the subject is a human being. In another embodiment, the animal is a non-human mammal, including laboratory research animals, pets, or livestock animals (e.g., mice, rats, non-human primates, cats, dogs, cows, horses, sheep, goats, pigs, or rabbits). In another embodiment, the animal is a bird.

Alternatively or additionally, the injection system includes a mechanism which is generally configured to control velocity of a product being moved through the injection system, for example, velocity of the product being moved through a cannula of the injection system, thereby reducing shear stresses on the product.

In some embodiments, the injection system includes mechanisms that control both pressure and velocity.

More specifically, the stress control mechanism provides an indicator for indicating when additional force on the plunger is appropriate, and providing a warning to the user when stress, resulting from excessive pressure and/or velocity, exceeds a safe level. The mechanism may further include a feature, for example, a hard stop feature, for preventing the user from causing the system to exceed a maximum allowable stress within the product. Other aspects are directed to enabling detection of a clog in a syringe during injection. Advantageously, in some embodiments, mechanisms are provided which can be readily incorporated into existing manual injection techniques (e.g., syringes, cartridges, auto injectors, etc.).

An excess of either normal stress or shear stress can lead to product (e.g., cell) damage. Normal stress is a function of force on the back of the plunger. By limiting the force applied to the plunger, both normal and shear stresses can be controlled, thereby preventing or reducing cell damage.

The injection systems and devices described herein may be configured to detect and/or control any desired range of pressures, depending on the nature of the product to be injected. For example, the maximum allowable pressure (e.g., a “red zone” pressure, as described elsewhere herein) at which an indicator signals to the user that the threshold has been reached, and/or at which the stress control mechanism prevents further pressure from being applied, may be about 5-10, 10-15, 15-20, 20-25, 25-30, 30-25, 35-40, 40-45, 45-50, 5-15, 10-20, 15-25, 20-30, 25-35, 30-40, 35-45, 40-50, 5-20, 10-25, 15-30, 20-35, 25-40, 30-45, or 35-50 psi, or about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50 or more psi. In some embodiments, for example, in a syringe system designed for injection of adipose (fat) cells, the maximum allowable pressure is about 10-20 psi, about 14-16 psi, or about 15 psi.

The injection systems and devices described herein may be constructed from any appropriate material known to those of skill in the art, and are preferably compatible with clinical practice and application to living subjects. Materials that may be used include, but are not limited to, plastic, rubber, glass, metal, and combinations thereof. The systems may be provided in sterile packaging (e.g., “ready-to-use”) or may be sterilizable by the user (e.g., using heat, irradiation, or chemical sterilization). In some embodiments, the systems are configured for single use (i.e., are disposable). Single use systems may protect subjects against cross-contamination or infection. Costs may be controlled by constructing single use systems from inexpensive materials such as plastic. In other embodiments, the systems are constructed for multiple uses. Multiple use systems may be preferred, for example, when more expensive materials are required for construction (and it thus would be cost-prohibitive to limit each system to a single use), or when the same device is being used to inject the same subject multiple times (such that cross-contamination and/or infection may not be a concern).

The injection systems and devices described herein may further be used in methods for administering an injectable substance to a subject comprising the steps of: loading the injectable substance (e.g., adipose cells) into an injection device as described herein, and using the device to inject the injectable substance into the subject. Where appropriate (e.g., when using a passive stress control mechanism), the stress control mechanism is monitored by the user during the injection procedure to ensure that the stress or pressure within the device does not exceed a predetermined level that would risk damage to the injectable substance (e.g., to living cells).

If the pressure reaches or exceeds the predetermined level, the injection procedure is slowed or stopped until the pressure decreases below the predetermined level, after which the injection procedure may be resumed (in embodiments using an active stress control mechanism, the system will act to slow or stop the procedure, or to release the excess pressure, on its own).

In some embodiments, the user may wait until the pressure decreases on its own before resuming the injection procedure. In other embodiments, the user may act to decrease the pressure. For example, if the system or device has become clogged, the user may act to remove the clog. Preferably, sterile conditions are used so as to prevent introduction of contamination or infectious agents.

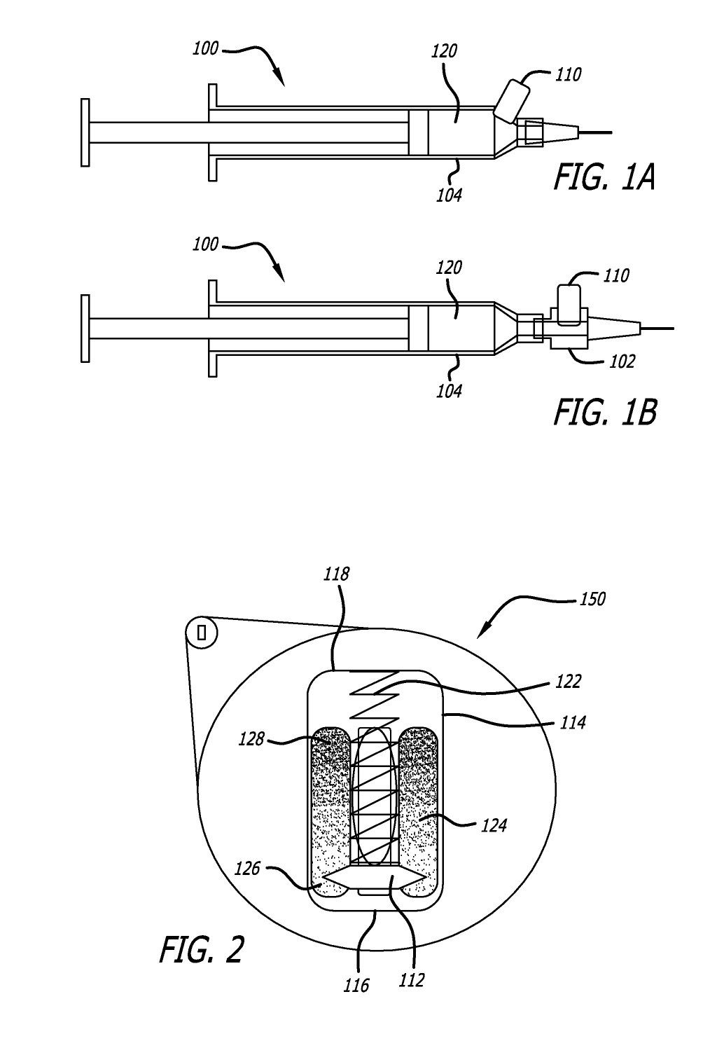

In one exemplary embodiment illustrated in FIGS. 1A-1B, a stress control mechanism comprises a visual feedback indicator device. As shown in FIG. 1A, the indicator device 110 may be located on the injector system 100 itself, or it may be a part of an accessory 102 to an existing injector device (FIG. 1B). The indicator device 110 may be configured to be in contact with or have access to the product 120 being injected, for example, either directly or through a membrane on the barrel 104.

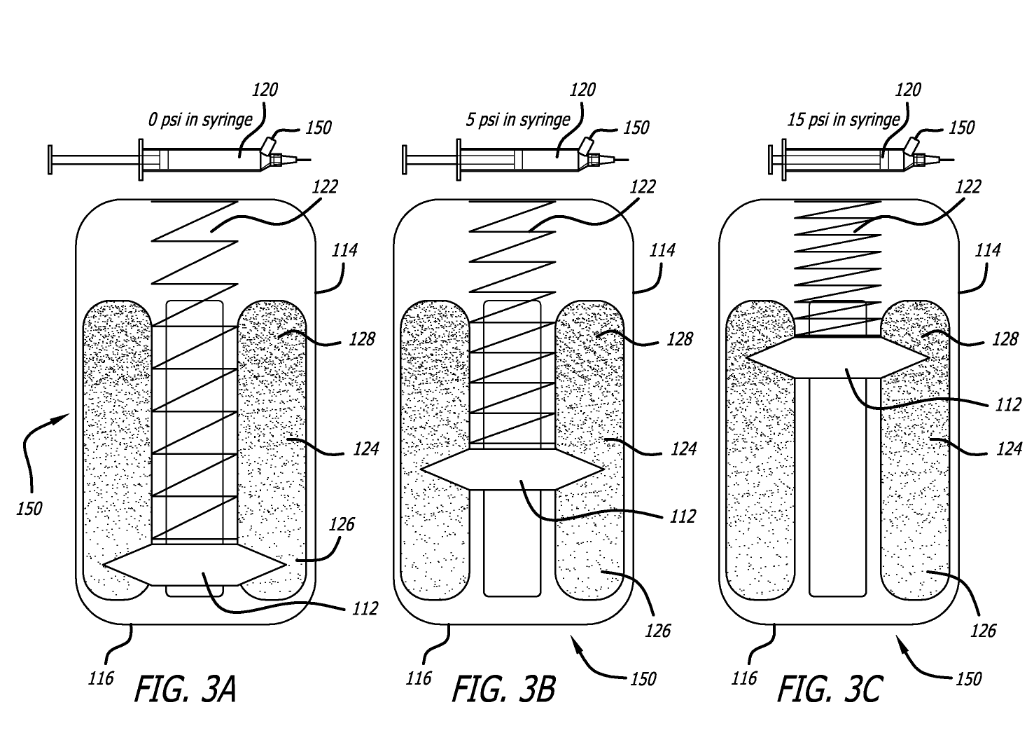

In one example, as illustrated in FIGS. 2 and 3A-3C, the indicator device 150 includes a spring-loaded plunger 112 disposed in a cylinder 114, the cylinder 114 having one end 116 in communication with fluid pressure in the syringe and another end 118 providing a visual indicator. The plunger 112 is moved in the cylinder 114 by a spring 122, for example, and provides a pressure reading based on the position of the plunger 112. The plunger 112 is attached to or includes an indicator 124 that is viewable by a user of the injector system. The indicator may be located on the exterior of the device. The plunger 112 may be, for example, forced down by the spring 122 within the cylinder 114 (FIG. 3A) and forced upward by pressure of the fluid (FIGS. 3B-3C). Displacement of the plunger 112 therefore may be used to provide a visual indication, or a reading, corresponding to a pressure within the device. In some embodiments, the amount of pressure in the system is indicated by intensity of a color, for example, with less intense color 126 representing an acceptable level of stress (FIG. 3B), or no excess stress (FIG. 3A), on the product, and more intense color 128 representing higher than acceptable stress, or excess stress (FIG. 3C), on the product, based on the amount of pressure detected in the syringe. In some embodiments, the amount of pressure in the system may be indicated by a color spectrum, for example, with green representing an acceptable level of stress, or no excess stress, on the product, and red representing higher than acceptable stress, or excess stress, on the product, based on the amount of pressure detected in the syringe.

In some embodiments, the visual indicator comprises a clog detector. For example, FIGS. 3A-3C illustrate how the indicator 150 changes as additional pressure is generated in the system 100. If, using this indicator, the product 120 gets clogged during injection, the user will gradually generate more and more force (FIG. 3B) until it he or she starts putting the product in jeopardy (dark color 128) (FIG. 3C). If the user reaches the “red zone” for this particular product and there is still no flow, it is safe to assume there is a clog. In embodiments using a single color, the “red zone” may refer to the darkest or most intense level of color 128. In embodiments using a single color, the “green zone” may refer to the lightest or least intense level of color 126. In embodiments using a color spectrum including multiple colors (e.g., green and red, although any desired colors may be used), the “red zone” may actually be red in color, while the “green zone” may actually be green in color.

The visual indicator device may be configured to provide a continuous indication to the user. In other words, the user can tell how close he or she is to the maximum allowable stress within the product 120 (red zone), even before it is reached, during the injection procedure. Use of a continuous indicator device allows continuous monitoring of the injection so to ensure that the indicator stays within the safe zone (or green zone in this case) during an entire injection procedure.

In some embodiments, while this visual indicator device is useful for indicating to the user when excess stress is being generated on the product, it may not prevent the user from generating those forces. In other embodiments, mechanisms may be provided for preventing a maximum allowable stress from being exceeded, as described in further detail below.

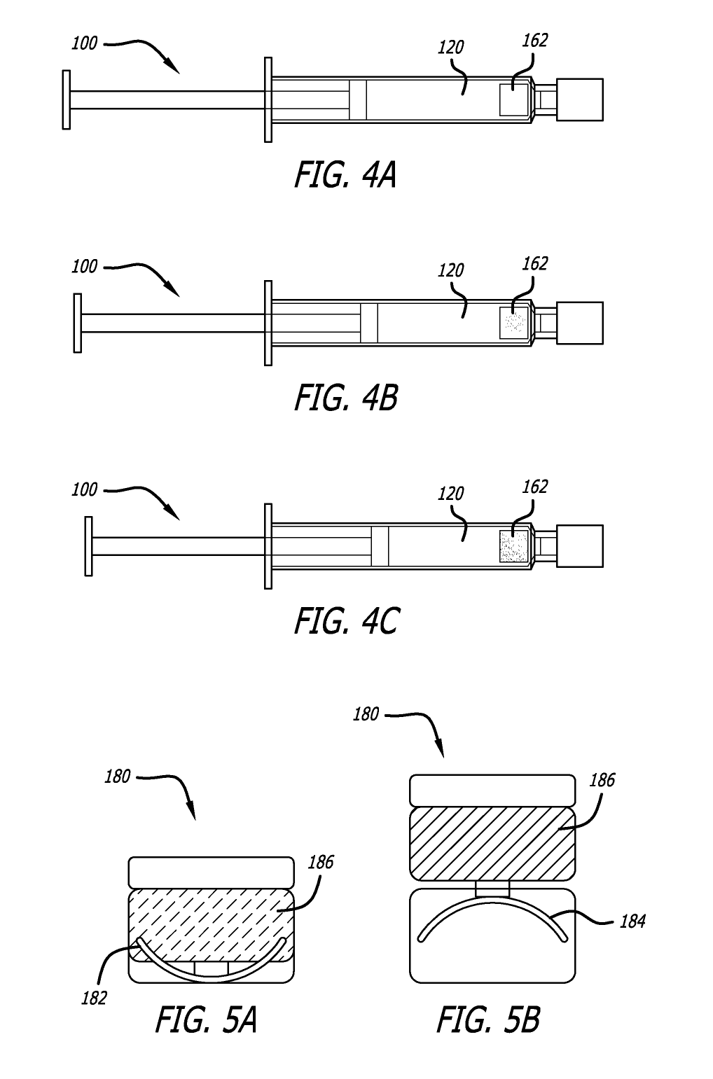

In one embodiment, the stress control mechanism comprises a photoelastic indicator device 162, as shown in FIGS. 4A-4C. Photoelasticity is a visual technique for measuring stresses. When a photoelastic material is strained and viewed under polarized light, a colored pattern can be observed. This colored pattern provides information on the stress state of the strained material. As illustrated in FIGS. 4A-4C, a piece of photoelastic material may be used (e.g., as part of the injector/syringe 160 or as an accessory) between two polarizers so as to reveal the presence of stresses due to pressure.

In another embodiment, a visual indicator is configured as a discrete indicator, rather than a continuous indicator as described above. A “discrete indicator”, as used herein, refers to an indicator that provides feedback to the user as discrete configurations. For example, in one embodiment, a discrete indicator may have two configurations: a “safe” or “go” configuration, indicating that it is safe for the user to proceed with the injection procedure, and an “unsafe” or “stop” configuration, which signals to the user that the stress or pressure within the system has exceeded a certain level and would risk damaging the injectable substance if the injection procedure were to continue.

Similar to the continuous visual feedback indicator described above, the discrete visual feedback indicator device can be either a part of an injector syringe system, or can be a separate accessory to an existing injector syringe (for example a luer connector that attaches between a syringe and needle).

In some embodiments, a discrete indicator mechanism is actuated by a “snap” mechanism. This snap may be any suitable material, for example, metal, plastic, rubber, or some sort of membrane, but it has the characteristic that it operates in a binary fashion, and is stable in only two states.

In one embodiment, as illustrated in FIG. 5A, a snap visual indicator 180 begins in a concave (up) orientation 182. Only after sufficient pressure exists below the snap 180 will it invert and “snap” to the concave (down) orientation 184 (FIG. 5B). In doing so, the snap pushes on indicator component 186, which indicates to the user that a specific pressure was reached. In one embodiment, the indicator component 186 may be visually distinctive (e.g., having a pattern or a color such as red or any other suitable color).

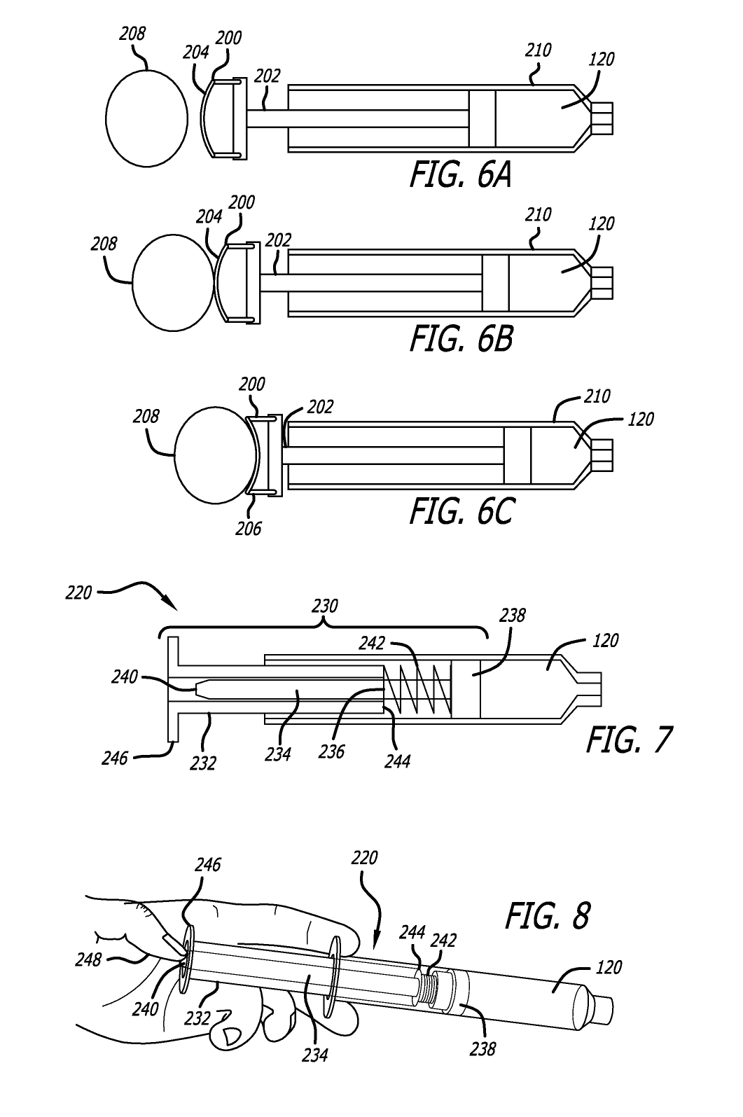

In one embodiment, as illustrated in FIGS. 6A-6C, a snap visual indicator 200 is placed at the rear of the syringe plunger 202 (FIG. 6A). The snap 200 holds its nominal position 204 (FIG. 6B) until a predetermined amount of force applied by the user 208 induces it to snap 206 (FIG. 6C), providing feedback to the user 208. Once this force is removed, the snap may revert 204 and the feedback is reset. Sufficient force to induce a snap 206 occurs when the pressure inside the syringe barrel 210 exceeds a certain predetermined level.

In some embodiments, a snap indicator as described above may make an audible sound when a predetermined amount of pressure is reached or exceeded, or when a predetermined amount of force is applied by the user. In other embodiments, the snap indicator may provide tactile feedback (as further described below).

In another exemplary embodiment, the stress control mechanism is configured to provide tactile feedback to the user, based on the pressure within the system. As illustrated in FIGS. 7-9, in the case of a syringe 220, it may comprise a plunger assembly 230 comprising a plunger rod 232 and a plunger tip 234 extending through the plunger rod 232, for example, through a center bore 236 in the plunger rod 232.

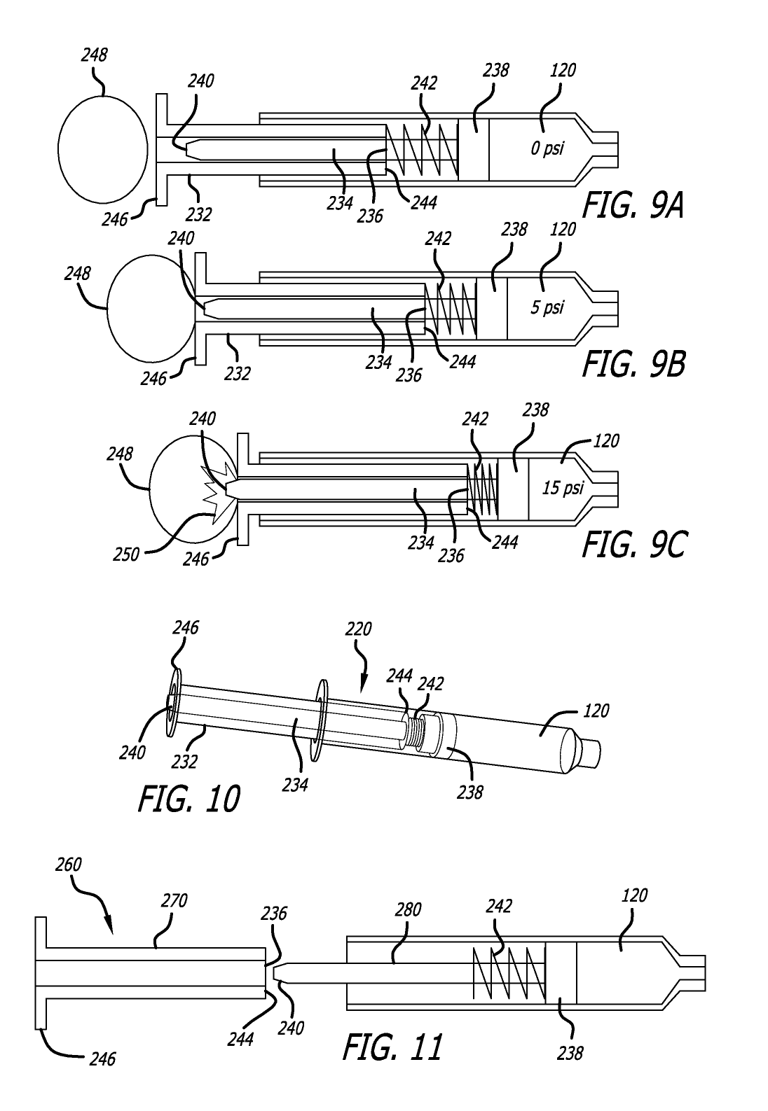

The plunger tip 234 may include a distal end including a head 238, and proximal end including a tactile indicating element 240. The plunger tip 234 is movable within the plunger rod 232, but movement is limited by a spring 242, for example, disposed between the head 238 and a distal portion 244 of the plunger rod 232. For example, the front (distal end) 244 of the plunger rod 232 and the distal end (head) 238 of the plunger tip 234 may be separated by a spring 242. The spring 242 has a spring constant or resilience that dictates at what pressure on the plunger tip 234 the user will receive feedback, thus indicating excess stress in the product 120 during injection. Such feedback may be provided by means of the proximal tactile indicating element 240 extending beyond the proximal end 246 of the plunger rod 232, and contacting the thumb 248 or finger of the user, such as illustrated in FIGS. 8 and 9C.

For example, as illustrated in FIGS. 9A-9C, in use, as the plunger 232 is depressed, it transmits a force to the spring 242, which in turn acts on the plunger tip head 238, ultimately creating a pressure that drives the product 120 out of the syringe 220. In order to generate additional pressure (to increase the flow rate of the product 120 for example), the user has to increase the force on the plunger 232, which is further transmitted through the spring 242 to the plunger tip 234 (FIG. 9A-9B). If there comes a point where the force being transmitted through the plunger 232 compresses the spring 242 to a point where the proximal end 240 of the plunger tip 234 extends past the proximal end 246 of the plunger 232 itself, the user will receive tactile feedback 250 as the proximal end 240 of the plunger tip 234 contacts the thumb 248 or finger of the user (FIG. 9C). The design of the proximal portion 240 of the plunger tip 234 is such that the user can readily determine when that instant happens. In the embodiment depicted in FIGS. 9A-9C, the proximal portion 240 of the plunger tip 234 is shaped as a tapered point. In other embodiments, it may alternatively be shaped as a dimple, plane, rod, textured region, or other geometry that is easily tactilely discernible by a user. A process for using this embodiment is depicted in FIGS. 9A-9C and 10.

In some embodiments, the plunger rod is easily separable from the plunger tip. FIG. 11 illustrates an embodiment where the plunger rod 270 and the plunger tip 280 are not securely or permanently connected. As shown in FIG. 11, the plunger tip 280 is not affected if the plunger rod 280 is removed therefrom. This feature may be useful in ensuring single use of the injection system 260, as it makes reusing the device more difficult if the plunger tip 280 cannot be easily or directly reset.

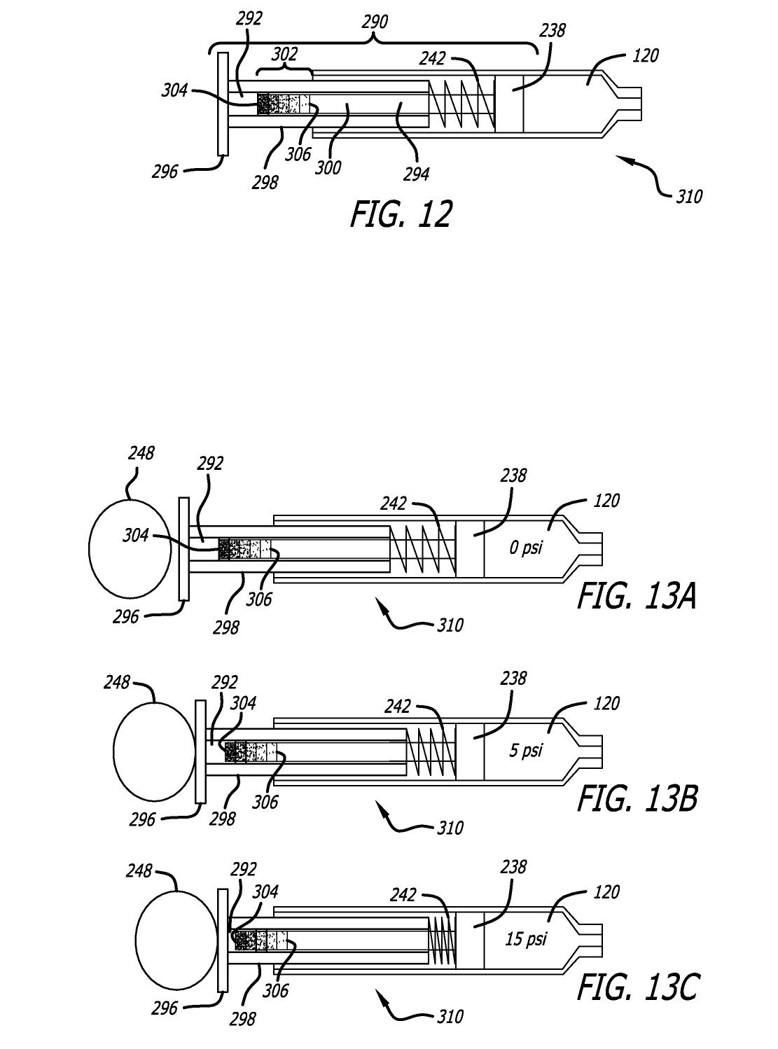

Another embodiment is illustrated in FIGS. 12 and 13A-13C. In this embodiment, the plunger assembly 290 is configured such that the proximal end 292 of the plunger tip 294 cannot extend past the proximal end 296 of the plunger rod 298. Additionally, the plunger tip shaft 300 may be labeled in a way that allows the user to visually, rather than tactilely, determine what pressure the product 120 is being subjected to. For example, in one embodiment, the plunger tip shaft 300 may be labeled with an indicator 302 comprising a single color, where the “red zone” may refer to the darkest or most intense level of color 304, while the “green zone” may refer to the lightest or least intense level of color 306. In embodiments using a color spectrum including multiple colors (e.g., green and red, although any desired colors may be used), the “red zone” may actually be red in color, while the “green zone” may actually be green in color.

For example, allowable pressure is indicated by “green zone” (less intense color or green color indicating less than maximum allowable pressure), and the “red zone” is indicated when maximum allowable pressure is reached or exceeded. FIG. 12 illustrates how this mechanism may look in a syringe 310.

FIGS. 13A-13C illustrate how this embodiment may work as a stress control mechanism under an increasing pressure load. It can be appreciated that the color indication could be replaced with any other suitable indicator, including explicitly indicating the pressure (e.g., with number values) within the system.

In another embodiment, one or more features of the two previously described embodiments in FIGS. 7-13, can be combined so that visual, continuous feedback is provided, and when the force becomes too high, tactile feedback is given in addition to the visual feedback.

In other exemplary embodiments, the stress control mechanism comprises a hard stop mechanism for preventing excess stress in the product from being reached. This feature is directed at preventing or stopping movement of the plunger once a certain pressure level or stress level is detected. These mechanisms essentially act as a “governor” or “restrictor” to the amount of stress that can exist within the product.

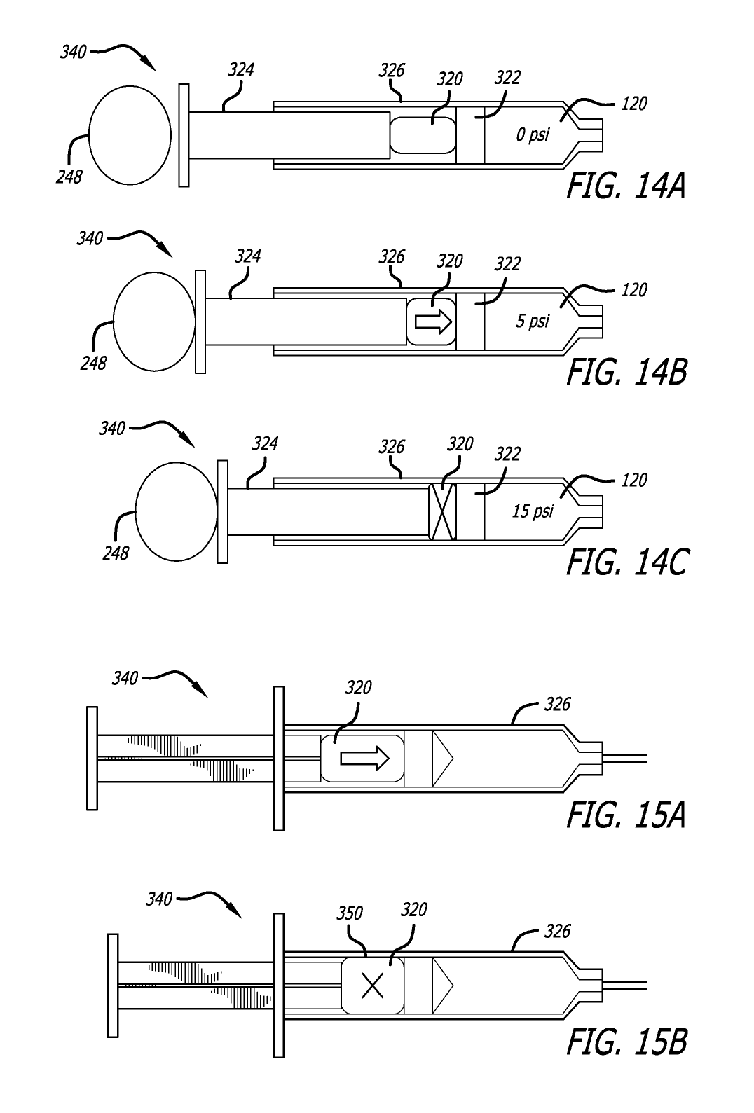

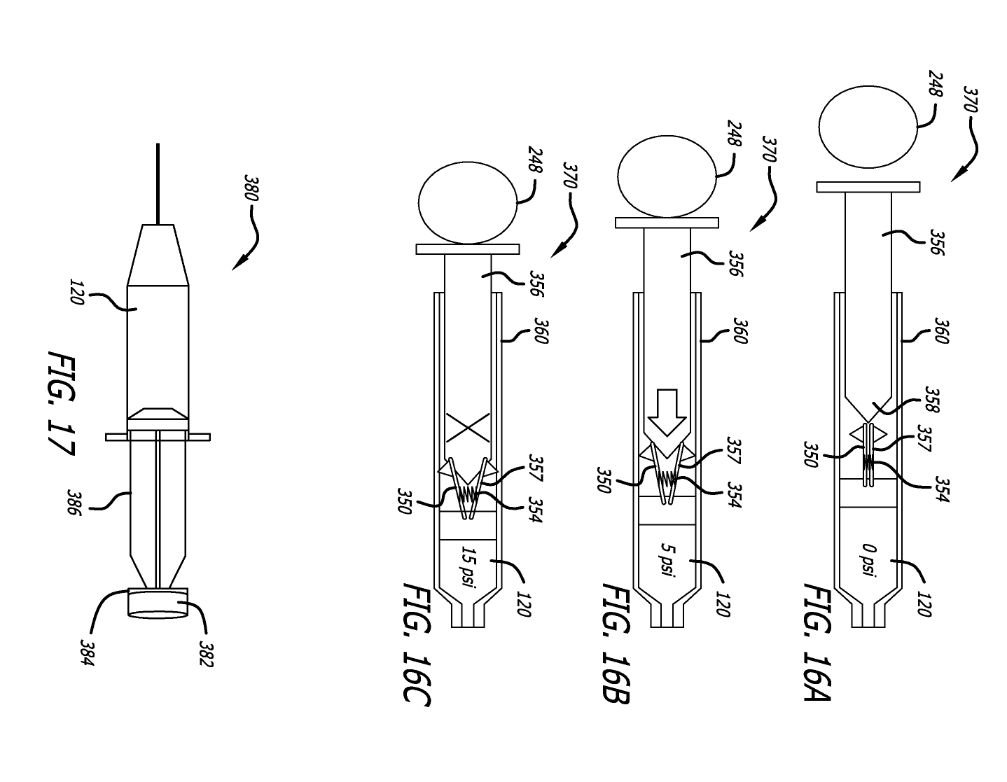

In one embodiment, illustrated in FIGS. 14A-14C and 15A-15B, a bumper 320 between the plunger tip 322 and the plunger rod 324 is provided. The bumper 320 can be tubular, cylindrical, or any other geometry that maximizes friction force against the syringe walls 326 when activated (FIG. 14C). In this mechanism, the bumper 320 is a material with a high coefficient of friction against the syringe barrel 326 material. It is also smaller than the syringe barrel 326 in its nominal state (FIG. 14A). As the user exerts a force against the plunger rod 324, the bumper 320 deforms and slightly increases in cross section 328 (FIG. 14B). Eventually, the user 248 may exert enough force (and therefore start to approach the maximum allowable stress within the product 120) such that the bumper 320 exerts a normal force 330 against the walls 326 of the syringe 340 (FIG. 14C). This normal force 330 is directly proportional to the frictional force generated. The bumper 320 material, geometry, and deformation is selected such that at the frictional force exceeds the force the user is exerting on the plunger rod 324. Ultimately, this means that the plunger rod 324 (as well as the plunger tip 322) cannot be moved forward. As the bumper 320 relaxes, either by release of pressure on the product side or by release of thumb or finger pressure by the user, its cross section gets smaller and the plunger rod 324 and tip 322 can be moved forward again.

Additionally, as illustrated in FIGS. 15A-15B, visual feedback may be provided to the user when the walls of the bumper 320 touch the walls 326 of the syringe 340 and are viewable to the user compressed against the inside walls 326 of the syringe 340 (FIG. 15B).

As described above for other embodiments, the stress control mechanism described above can be optimized to ensure single use, for example, by providing certain elements as physically separate pieces (e.g., by providing the plunger rod 324 and the bumper 320 as non-physically-linked parts), or by other means.

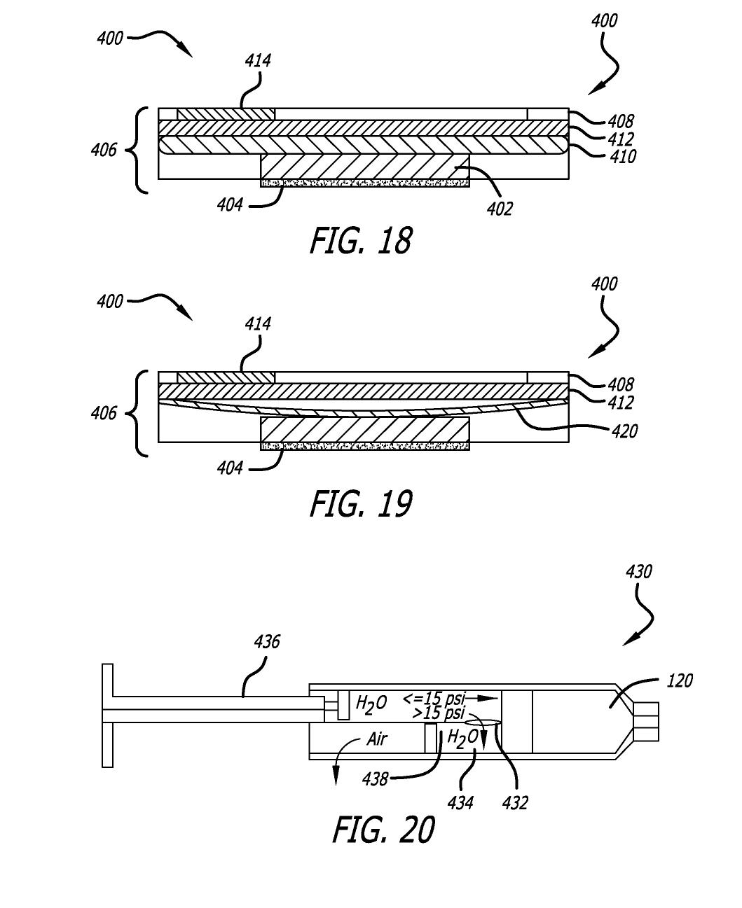

The hard stop feature may alternatively comprise solid components that do not depend not on deformation, but on linkage motion, for example. An example is illustrated in schematic in FIGS. 16A-16C. In this embodiment, a series of several mechanical linkages 350 is attached to the plunger tip 352 (FIG. 16A). The linkages 350 are drawn toward the center of the syringe 370 by springs 354 (FIG. 16A). As the user presses the plunger rod 356 forward, the front 358 of the plunger rod 356 pushes all the linkages 350 out toward the syringe barrel walls 360 (FIG. 16B). Eventually, the force exerted by the user may become so large that the linkages 350 are driven into the surface of the syringe barrel walls 360, forcing the system 370 to stop (FIG. 16C). Additionally, there may or may not be features that are part of the syringe barrel 360 that aid in creating a mechanical stop once the linkage components 350 are in contact.

In another embodiment, a slip or clutch mechanism may be used to prevent the user from exerting too much force. For example, a plunger rod and plunger tip which are two independent components can be configured to provide a slip or clutch mechanism. Under a specific force, the slip/clutch acts as a rigid, unitary component. Once the maximum allowable stress within the system is achieved, however, the plunger rod is allowed to slip past the plunger tip component so that no further stress can be exerted onto the product. Note that the system may be reset when all force is removed via a spring or some other return mechanism (so that pushing can continue).

In another embodiment, FIG. 17 illustrates an electro-mechanical based mechanism 382 for measuring the pressure applied to the product 120 within a syringe 380 during extrusion or injection. In this embodiment, an electromechanical sensor and indicator 382 is an assembly that can be secured to an end 384 of a plunger 386 of a conventional syringe 380, and is configured to sense pressure against the plunger 386 by a user.

The electronic sensor and indicator 382 may be attached to the proximal end 384 of the plunger 386 that is pushed, typically by the finger or thumb, to extrude the product 120 within the syringe 380. As the device is pushed to advance the plunger 386, the applied force is sensed. If the applied force exceeds a predetermined threshold, then an indicator 382 is activated. In one embodiment, the indicator is the illumination of a light emitting diode (LED) or multiple LEDs. Alternative indicators include an audible tone or sound, or haptic vibration that can be implemented individually or in combination. Conversely, if the applied force is below a predetermined threshold, no indicator is activated.

The housing of the electronic indicator may be fabricated from any suitable material, including, but not limited to, plastic (e.g., inexpensive plastic), metal, rubber, or ceramic. The housing can have suitable apertures to allow the indicator to be recognized. In some embodiments, the indicator device may be attached to the syringe plunger end using an adhesive, for example, an adhesive strip with a peel-away backing to preserve the adhesive until ready for use). Use of an adhesive in this manner allows attachment of the device to practically any type of syringe without modification. Other means of attachment include clips that attach to the edge of the plunger end, grooves that slide onto the end of the plunger, or a flexible skirt that wraps around the end of the plunger.

Details for the construction of an example of this device 400 are illustrated in FIG. 18. One construction utilizes a binary force sensor 402 acting as a switch with activation above a specific threshold and no activation below the threshold. The binary force sensor 402 may be constructed, for example, from spring steel similar to common electronic dome switches, or an actual spring of metal or plastic. The binary force sensor 402 can be continuously variable, making it possible to measure a range of forces. A variable sensor may use, for example, a piezo, resistive, or capacitive implementation measuring force or pressure as representative but not all-inclusive examples. A continuously variable force sensor makes it possible to ramp up the indicator, for example increasing LED brightness and/or change color from green to yellow, as the threshold is approached, and then show an alternate state, such as flashing the LED and/or changing color from yellow to red, when the threshold is exceeded.

The compliant interface 404 allows the force pushing on the plunger to translate from the end of the plunger to the binary force sensor 402. It can be, for example, a flexible membrane that is the same diameter as the device 400 or a smaller diameter with the device housing 406 providing a rigid support.

As mentioned previously, the indicator 408 can be an LED or other illumination device, a vibration device such as motor, or tone generator such as a speaker or piezo element.

The device 400 may further comprise a battery 410. The battery 410 may comprise generally a coin cell, for example, having a lithium chemistry for long shelf life and favorable voltage. The size of the battery 410 is not critical, but preferably is of a diameter that is the same or smaller than that of a typical syringe plunger end. The battery 410 may have a width that is sufficiently thin in order to minimize impact to the injection procedure. Similarly, the capacity of the battery is not critical, as long as it can provide power for at least one injection procedure (for a disposable or single-use device). A reusable device would comprise a battery with capacity dictated by the number of intended injections.

The device 400 may further comprise a printed circuit board 412. At least in part, the printed circuit board 412 may serve as a substrate for the indicator 408, basic electronic components, and contact to one side of the battery 410. A flexible circuit or wire is connected from the printed circuit board to allow contact and complete the circuit with the other side of the battery 410. Similar to the battery 410, the circuit board 412 is ordinarily the same or smaller diameter of a typical syringe plunger end and as thin as possible. In one embodiment, the printed circuit board 412 can also provide interconnection for additional electronic components 414.

The basic electronic components 414 exist to support the force measurement and activate the indicator 408. More involved electronic components including, but not limited to, a micro-controller, can be incorporated to provide additional features such as support for a variable sensor and thus providing for changing LED intensity, color, illumination location, or flash rate. Alternatively or additionally, an audible indicator may be provided, for example, an indicator manifested in a changing tone, tone rate, tone sequence, or volume. Alternatively still, a vibration indicator may be provided with a changing speed, pulse rate, pulse sequence, or intensity. Another feature that may be supported by electronic components is a low battery indicator. Yet another feature that may be supported by electronic components is the ability to select a force threshold or syringe type (e.g., syringe volume, syringe brand) with a suitable usable interface such as a small button, for example, on the side of the device, comparable to the common reset button on electronic devices, with the chosen selection signified by the indicator. Yet another feature that may be supported by the electronic components is the ability to shut the device off, either to save power for multi-use or to enforce single use.

A single device type with a variable force sensor can be calibrated for any particular syringe size and force combination. Alternatively, multiple device types, each with a different binary force sensor, can be implemented such that each type is designated for a specific syringe and force combination. For example, a device may be calibrated or constructed to correspond to a 30 cc syringe and 25 psi extrusion force, while a different device may be calibrated or constructed to correspond to a 50 cc syringe and 30 psi extrusion force.

Alternatively, as illustrated in FIG. 19, a piezoelectric element 420 may be used not only as a sensor, but also as the power source, instead of a battery. The deformation of the piezoelectric element 420 would generate the power for the whole system.

An alternative embodiment may contain a valve system controlled by the amount of internal pressure of the syringe. The valve would either open or close, depending on the pressure in a first reservoir. During normal injection, the pressure would be transferred from the first reservoir, through the plunger, to a second reservoir (which would, in fat grafting applications, contain the fat), and ultimately extrude.

One example of this embodiment, illustrated in FIG. 20, is a syringe assembly 430 comprising a pressure relief valve 432, or a valve that opens when a pressure reaches a set point. In another example, a secondary, parallel reservoir 434 is provided which fills with fluid 438 (e.g., water or saline) when the pressure relief valve 432 opens. The valve 432 opens under pressure higher than the set point, which results from a force exerted by the user on the plunger 436.

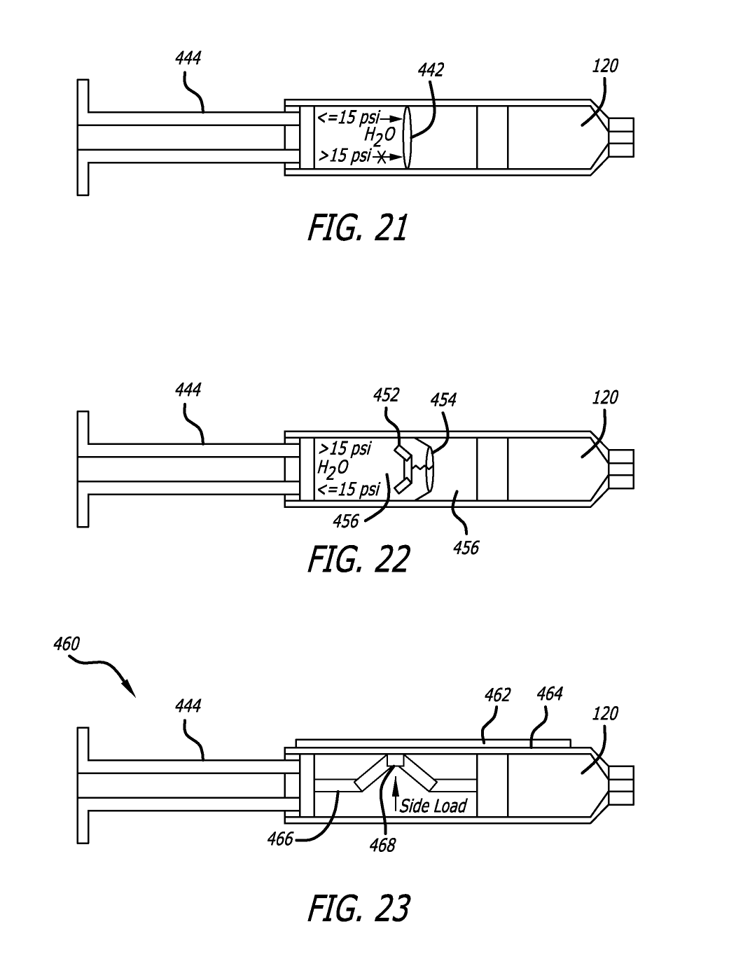

Another example of this embodiment, illustrated in FIG. 21, is a converse of the example shown in FIG. 20, in that a valve 442 closes at a certain pressure, thus preventing the internal pressure from transferring to the plunger 444.

Another embodiment, illustrated in FIG. 22, comprises a mechanical element 452 that blocks a hole 454 under a certain pressure. During normal operation, the mechanical element 452 suspends above the hole 454 and allows fluid 456 (e.g., water or saline) to pass through. When the pressure reaches a certain point, it blocks the hole 454, preventing fluid 456 from passing through.

In another embodiment, illustrated in FIG. 23, the stress control mechanism comprises a pressure film 462 alongside the syringe 460 that changes properties throughout the injection, resulting in a map, or profile, of injection force. This profile could be used to determine at what points during injection there was too much force, for how long, and how often.

Pressure films are commercially available, and are useful in this embodiment. One such commercially available film contains colored microcapsules that burst under high surface pressure. Higher pressures are indicated by a relatively more apparent color on the film. In one embodiment, the film is placed in the inside of a clear syringe barrel 464 and provides a profile of different shades of a color to produce an injection force graph. In order to induce pressure on the film, a plunger rod 466 may be provided which includes a suitable structural feature 468 that translates injection force to side load against the wall 464 of the syringe 460.

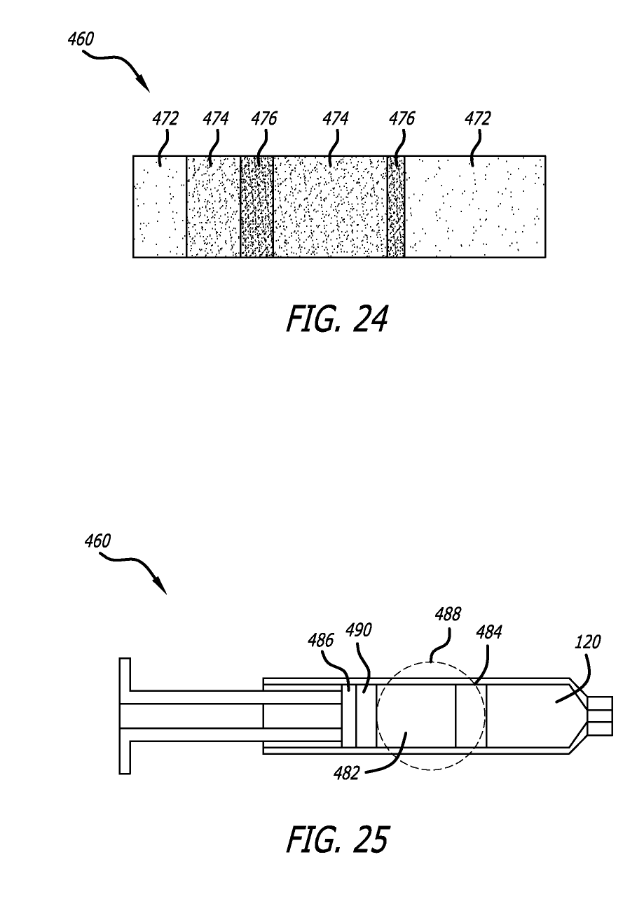

An example of an injection profile in a pressure film 462 after an injection procedure using the system illustrated in FIG. 23 is illustrated in FIG. 24. The lighter 472 and medium 474 shaded areas correspond to light and medium force exertions, respectively, whereas the darker 476 areas are points at which too much force was exerted, and possibly, for example, damaging cells (e.g., fat cells) in an injectable composition.

In another embodiment, illustrated in FIG. 25, a compartment 482 of magnetorheological (MR) fluid is positioned in the syringe barrel 484, the compartment of fluid acting as a stopper during high force injections. MR Fluid is normally an oil-like consistency that, under a magnetic field, changes properties and becomes significantly more viscous, and would become almost impossible to push in a standard syringe 480. The viscosity varies depending on the intensity of the applied magnetic field. Means to apply a magnetic field may be provided, as well as a means to sense when injection force is too high. One example is a force sensor 486 or dome switch that, when activated, turns on an electronic circuit 490 that applies a small, local magnetic field 488.

Alternatively, a shear thickening fluid (dilatant) may be used. This material has the property such that it will eventually normalize and achieve equilibrium. For example, if shear increases in the shear thickening fluid, its viscosity increases, making it more difficult to push. However, as the ability to push decreases, so does the velocity (and therefore shear), causing the viscosity to decrease (and allowing pushing to continue).

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. An injection device comprising;
a syringe comprising a barrel suitable to contain an injectable substance, and a plunger, movable within the barrel for forcing the injectable substance through the a distal end of the syringe, and
a stress control mechanism configured to detect, control, or detect and control a pressure within the barrel to reduce potential for damaging the injectable substance.

2. The injection device of claim 1, wherein the stress control mechanism is a visual feedback indicator configured to detect a pressure within the barrel and display visual feedback to a user, wherein the visual feedback indicator is selected from a continuous visual feedback indicator and a discrete visual feedback indicator.

3. The injection device of claim 2, wherein the visual feedback indicator comprises:
a cylinder having a distal end in communication with fluid pressure in the barrel,
a spring-loaded plunger movable within the cylinder, and
an indicator configured to display a pressure reading based on the position of the plunger.

4. The injection device of claim 2, wherein the visual feedback indicator comprises photoelastic material disposed between two polarizers.

5. The injection device of claim 2, wherein the visual feedback indicator comprises a binary snap indicator configured to maintain one configuration when the pressure is below a predetermined level and to change to a second configuration when the pressure reaches or exceeds the predetermined level.

6. The injection device of claim 1, wherein the stress control mechanism is a tactile feedback indicator configured to detect a pressure within the barrel and provide tactile feedback to a user.

7. The injection device of claim 6, wherein the tactile feedback indicator comprises a plunger assembly comprising:
a plunger rod comprising a distal end and a proximal end;
a plunger tip extending through the plunger rod, wherein the plunger tip comprises a distal end comprising a head and a proximal end comprising a tactile indicating element;
a spring disposed between the plunger tip head and the distal end of the plunger rod;
wherein the plunger tip is movable within the plunger rod.

8. The injection device of claim 2, wherein the visual feedback indicator comprises a plunger assembly comprising:
a plunger rod comprising a distal end and a proximal end;
a plunger tip extending through the plunger rod, wherein the plunger tip comprises a distal end comprising a head and a proximal end comprising an indicator configured to display a pressure reading based on the position of the plunger tip;
a spring disposed between the plunger tip head and the distal end of the plunger rod;
wherein the plunger tip is movable within the plunger rod.

9. The injection device of claim 1, wherein the stress control mechanism comprises a hard stop mechanism configured to prevent the pressure within the barrel from exceeding a predetermined level.

10. The injection device of claim 9, wherein the plunger comprises a distal end and a proximal end, and wherein the hard stop mechanism comprises a deformable bumper disposed between the proximal end of the plunger and the distal end of the plunger, and wherein the deformable bumper is configured to exert a frictional force against the barrel when the pressure within the barrel exceeds a predetermined level, and wherein the frictional force prevents the plunger from moving within the barrel.

11. The injection device of claim 9, wherein the hard stop mechanism comprises a plunger assembly comprising:
a plunger rod comprising a distal end and a proximal end;
a plunger tip disposed within the barrel;
a series of mechanical linkages connected by one or more springs disposed between the distal end of the plunger rod and the plunger tip;
wherein the plunger assembly is movable within the barrel when the pressure within the barrel is below a predetermined level; and
wherein the mechanical linkages prevent movement of the plunger assembly by contacting the barrel when the pressure within the barrel reaches or exceeds the predetermined level.

12. The injection device of claim 1, wherein the stress protection mechanism comprises an electromechanical sensor and indicator, wherein the electromechanical sensor and indicator is configured to produce a visual, aural, tactile, or vibrational indication when the pressure within the barrel reaches or exceeds a predetermined level.

13. The injection device of claim 12, wherein the electromechanical sensor and indicator comprises at least one light emitting diode (LED).

14. The injection device of claim 12, wherein the electromechanical sensor and indicator comprises: electronic components, a printed circuit board, a compliant interface, a sensor, an indicator, and a battery or piezoelectric element.

15. The injection device of claim 9, wherein hard stop mechanism comprises a plunger assembly comprising:
a plunger rod comprising a distal end and a proximal end;
a plunger tip disposed within the barrel;
a compartment disposed between the distal end of the plunger rod and the plunger tip, wherein the compartment comprises magnetorheological fluid; and
a force sensor and an electronic circuit disposed between the distal end of the plunger rod and the compartment;
wherein the electronic circuit produces a magnetic field when the force sensor detects a pressure within the barrel that reaches or exceeds a predetermined level.

16. The injection device of claim 1, wherein the stress control mechanism comprises a pressure release valve configured to open and release pressure when the pressure within the barrel reaches or exceeds a predetermined level, or a reverse pressure release valve configured to close when the pressure within the barrel reaches or exceeds a predetermined level.

17. The injection device of claim 1, further comprising a second stress protection mechanism configured to detect, control, or detect and control a pressure within the barrel to reduce potential for damaging the injectable substance.

18. The injection device of claim 1, wherein the distal end is connectable to a cannula, and further comprising a cannula disposed in the distal end of the syringe.

19. The injection device of claim 1, further comprising an injectable substance within the barrel.

20. A method for administering an injectable substance to a subject comprising the steps of:
loading the injectable substance into the injection device of claim 1;
pushing the plunger within the barrel to inject the injectable substance into a target site in the subject; and
stopping the pushing when the stress control mechanism indicates that a predetermined level of pressure in the barrel is reached, or when the stress control mechanism prevents further movement of the plunger within the barrel.
